(19) United States
(12) Patent Application Publication
(10) Pub. No.: US 2006/0270658 A1
(43) Pub. Date: Nov. 30, 2006

(54) Use of oxcarbazepine for the treatment of diabetic neuropathic pain and the improvement of sleep

(76) Inventor: Donald Manning, Bloomsbury, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/570,340

(22) PCT Filed: Sep. 2, 2004
(86) PCT No.: PCT/EP04/09797

§ 371 Date: Jun. 21, 2006

Related U.S. Application Data
(60) Provisional application No. 60/537,378, filed on Jan. 16, 2004.

(30) Foreign Application Priority Data
Sep. 3, 2003 (GB) 0320637.2

(51) Int. Cl. A61K 31/55

(52) U.S. Cl. 514/217

(57) ABSTRACT

The present invention provides for novel uses of the carbamazepine derivative of formula (I) 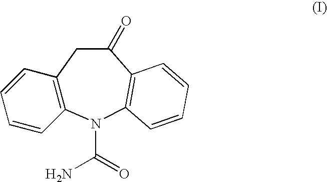 and its pharmaceutically acceptable salts, in particular an improved regiment for the administration of the carbamazepine derivative of formula (I) and the pharmaceutically acceptable salts thereof for the treatment of patients suffering from pin, in particular neuropathic paid, and the improvement of sleep; the use of oxcarbazepine for the manufacture of a pharmaceutical composition for the treatment of pain or the manufacture of a pharmaceutical composition for the improvement of sleep in human patients suffering from chronic pain; pharmaceutical composition comprising oxcarbazepine as sole active ingredient for the treatment of pain or for the improvement of sleep in human patients suffering from chronic pain and to packages comprising a pharmaceutical composition comprising as sole active ingredient oxcarbazepine together with instructions for the treatment of pain or together with instructions for improvement of sleep in human patients suffering from chronic pain.

The present invention relates to novel uses of the carbamazepine derivative of formula I 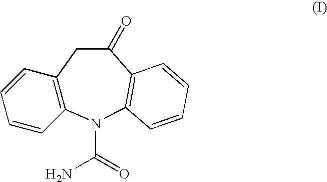
and its pharmaceutically acceptable salts, in particular an improved regimen for the administration of the carbamazepine derivative of formula I and the pharmaceutically acceptable salts thereof for the treatment of patients suffering from pain, in particular neuropathic pain, especially diabetic neuropathic pain, and the improvement of sleep.

The compound of formula I is known as “oxcarbazepine” (10-oxo-10, 11-dihydro-5H-dibenz[b,f]azepine-5-carboxamide) and is, e.g., marketed under the brand name Trileptal®.

Oxcarbazepine is a known anticonvulsant drug useful in the treatment of seizures of, for example, epileptic origin. Its preparation is described, e.g., in the U.S. Pat. No. 3,642,775 and WO 01/56992, which are both herein incorporated by reference.

Surprisingly, it was found that an efficacious treatment of pain, in particular neuropathic pain, especially diabetic neuropathic pain, in human patients, in particular in a Caucasian patient population, can be achieved by administering oxcarbazepine according to one of the dosing regimens of the present invention. Even more surprisingly, an effect against neuropathic pain can be obtained already by using comparatively low daily doses of oxcarbazepine, e.g. 450 or 600 mg/day, i.e. at total daily doses being substantially lower than those which are commonly applied for the treatment of epilepsy.

Furthermore, it was surprisingly found that sleep can be improved in human patients suffering from chronic pain, in particular neuropathic pain, especially diabetic neuropathic pain, by decreasing the frequency of being awakened from sleep due to pain, decreasing the delay of getting to sleep due to pain or feeling rested after sleep by administering oxcarbazepine, especially according to one of the dosing regimens of the present invention.

According to one embodiment of the present invention, oxcarbazepine or a pharmaceutically acceptable salt thereof is administered, e.g. twice or more daily, for example two or three times daily, at a total dose in the range from about 450 mg/day to about 900 mg/day, particularly a total dose of about 550 mg/day to about 750 mg/day, especially 600 mg/day. Hence, the present invention relates to a method of administering oxcarbazepine to a patient, which comprises administering oxcarbazepine, or a pharmaceutically acceptable salt thereof, to a patient suffering from pain, in particular neuropathic pain, especially diabetic neuropathic pain, especially on a twice daily schedule, at a total dose in the range from about 450 mg/day to about 900 mg/day, particularly a total dose of about 550 mg/day to about 750 mg/day, especially 600 mg/day.

Furthermore, the present invention relates to a method of improving sleep in human patients suffering from chronic pain, in particular neuropathic pain, especially diabetic neuropathic pain, which comprises administering oxcarbazepine or a pharmaceutically acceptable salt thereof to said patient especially on a twice daily schedule, at a total dose in the range from about 450 mg/day to about 900 mg/day, particularly a total dose of about 550 mg/day to about 750 mg/day, especially 600 mg/day.

According to a further embodiment of the present invention, oxcarbazepine or a pharmaceutically acceptable salt thereof is administered, e.g. twice or more daily, for example two or three times daily, at a total dose in the range from about 900 mg/day to about 1500 mg/day, particularly a total dose of 1050 mg/day to 1350 mg/day, especially 1200 mg/day. Hence, the present invention relates to a method of administering oxcarbazepine to a patient, which comprises administering oxcarbazepine, or a pharmaceutically acceptable salt thereof, to a patient suffering from pain, in particular neuropathic pain, especially diabetic neuropathic pain, especially on a twice daily schedule, at a total dose in the range from about 900 mg/day to about 1500 mg/day, particularly a total dose of 1050 mg/day to 1350 mg/day, especially 1200 mg/day.

Furthermore, the present invention relates to a method of improving sleep in human patients suffering from chronic pain, in particular neuropathic pain, especially diabetic neuropathic pain, which comprises administering oxcarbazepine or a pharmaceutically acceptable salt thereof to said patient especially on a twice daily schedule, at a total dose in the range from about 900 mg/day to about 1500 mg/day, particularly a total dose of 1050 mg/day to 1350 mg/day, especially 1200 mg/day.

According to q further embodiment of the present invention, oxcarbazepine or a pharmaceutically acceptable salt thereof is administered, e.g. twice or more daily, for example two or three times daily, at a total dose in the range from about 1500 mg/day to about 2100 mg/day, particularly a total dose of 1650 mg/day to 1950 mg/day, especially 1800 mg/day. Hence, the present invention relates to a method of administering oxcarbazepine to a patient, which comprises administering oxcarbazepine, or a pharmaceutically acceptable salt thereof, to a patient suffering from pain, in particular neuropathic pain, especially diabetic neuropathic pain, especially on a twice daily schedule, at a total dose in the range from about 1500 mg/day to about 2100 mg/day, particularly a total dose of 1650 mg/day to 1950 mg/day, especially 1800 mg/day.

Furthermore, the present invention relates to a method of improving sleep in human patients suffering from chronic pain, in particular neuropathic pain, especially diabetic neuropathic pain, which comprises administering oxcarbazepine or a pharmaceutically acceptable salt thereof to said patient especially on a twice daily schedule, at a total dose in the range from about 1500 mg/day to about 2100 mg/day, particularly a total dose of 1650 mg/day to 1950 mg/day, especially 1800 mg/day.

The term “neuropathic pain” as used herein includes, but is not restricted to, pain that frequently accompanies nerve damage resulting from a range of pathologies including amputation or conditions such as diabetes, post-herpetic neuralgia or trigeminal neuralgia. The hyperalgesia and allodynia associated with neuropathic pain is particularly intractable and poorly treated in the clinic by treatments such as opiates or non-steroidal anti-inflammatory drugs.

The usefulness of oxcarbazepine in the treatment of the above-mentioned disorders including the improvement in sleep quality can be confirmed in suitable clinical studies, e.g. those described in the Examples, e.g. applying a total daily dosage of 600 mg, 1200 mg or 1800 mg oxcarbazepine. The person skilled in the pertinent art is fully enabled to select a relevant test model to prove such usefulness. Suitable clinical studies are in particular randomized, double-blind, placebo-controlled, parallel studies in neuropathic pain patients.

### SHORT DESCRIPTION OF THE FIGURES

FIG. 1 depicts the average VAS score (y-axis) during the last week of treatment of the clinical study described in Example 1. The darker area represents the scores obtained in the group of patients obtaining oxcarbazepine at a daily dosage of 1800 mg (N=68). The brighter area represents the score for the placebo group (N=76).

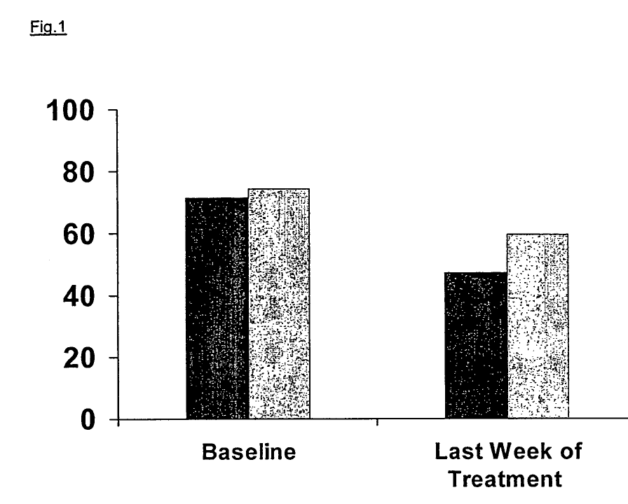

FIG. 2 illustrates the average VAS score (y-axis) by week (x-axis) of the clinical study described in Example 1. The upper lines represents the score for the placebo group (N=76). The lower line represents the scores obtained in the group of patients obtaining oxcarbazepine at a daily dosage of 1800 mg (N=68).

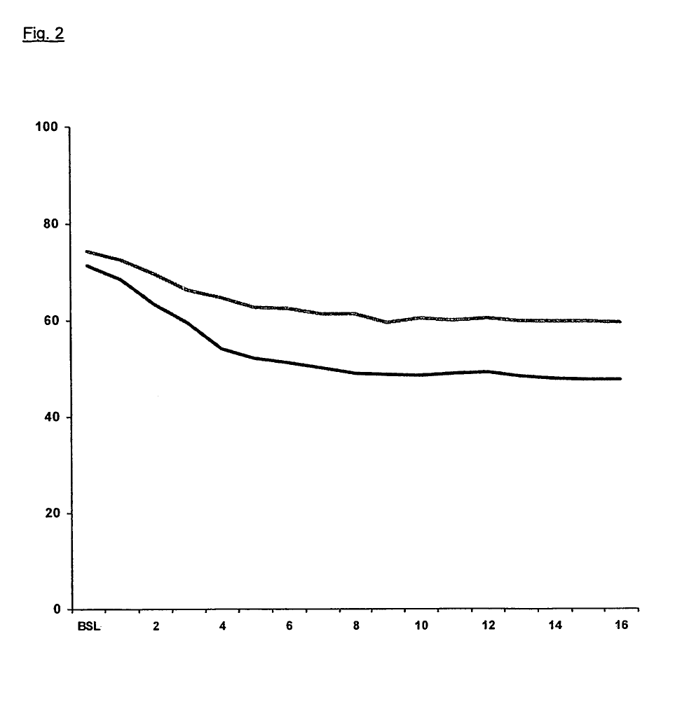

According to one aspect of the present invention, oxcarbazepine is given twice daily on a continuous basis, alone, or during and subsequent to other therapies, for example during the treatment of diabetes.

The single doses applied can range between 150 and 1200 mg, e.g. 300 mg, 600 mg or 900 mg, of oxcarbazepine. For instance, in one embodiment of the invention, two single doses of about 900 mg are applied 6 to 12 hours apart, for example about 8 hours apart.

Oxcarbazepine may be administered in any usual manner, e.g. orally, for example in the form of tablets or capsules, or parenterally, for example in the form of injection solutions or suspensions.

In one embodiment of the invention, the pharmaceutical composition is preferably a tablet, more preferably a tablet as disclosed in U.S. Pat. No. 4,353,887 or, most preferably, a film-coated tablet, e.g. as described in WO 98/35681, which publications are both incorporated herein by reference, especially the working examples.

In another embodiment of the invention, the pharmaceutical composition is preferably an oral suspension, more preferably an oral suspension as disclosed in WO 01/45671, which is incorporated herein by reference.

Unit dosage forms may contain, for example, from about 2.5 mg to about 1000 mg of oxcarbazepine, e.g. 150 mg or 300 mg.

The invention further provides the use of oxcarbazepine for the manufacture of a pharmaceutical composition for the treatment of pain, in particular neuropathic pain, especially diabetic neuropathic pain, in particular characterized in that the composition comprises between 300 and 1200 mg of oxcarbazepine.

The invention additionally provides the use of oxcarbazepine for the manufacture of a pharmaceutical composition for the improvement of sleep in human patients suffering from chronic pain, in particular neuropathic pain, especially diabetic neuropathic pain.

Furthermore, the present invention provides
a package comprising a pharmaceutical composition comprising as sole active ingredient oxcarbazepine together with instructions for improvement of sleep in human patients suffering from chronic pain, in particular neuropathic pain, especially diabetic neuropathic pain; and
a pharmaceutical composition comprising as sole active ingredient oxcarbazepine for the improvement of sleep in human patients suffering from chronic pain, in particular neuropathic pain, especially diabetic neuropathic pain.
a package comprising a pharmaceutical composition comprising as sole active ingredient oxcarbazepine together with instructions for the treatment of pain, in particular neuropathic pain, especially diabetic neuropathic pain; and
a pharmaceutical composition comprising as sole active ingredient oxcarbazepine for the treatment of pain, in particular neuropathic pain, especially diabetic neuropathic pain.

### EXAMPLE 1

### Clinical Study in Human Diabetic Neuropathic Pain Patients

In a clinical study in a Caucasian patient population, a full double-blind treatment was conducted for a period of 112 days. Patients suffering from diabetic neuropathic pain obtained placebo or oxcarbazepine. Oxcarbazepine at a total dosage of 1800 mg/day was statistically significantly superior to placebo with respect to the average Visual Analog Scale (VAS) for pain severity score during the last week of double-blind treatment (p=0.0108). Furthermore, the group treated with oxcarbazepine at a total dosage of 1800 mg/day had a statistically significantly higher percentage of responders (those felt slightly, much, or very much improved) (73.2%) compared with the placebo group (39.7%) (p=0.0003).

### EXAMPLE 2

### Impact of Oxcarbazepine on Sleep in Human Diabetic Neuropathic Pain Patients

In a multicenter, placebo-controlled, double-blind, parallel-group study the efficacy of oxcarbazepine up to 1800 mg/day in patients with neuropathic pain of diabetic origin was evaluated. The study consisted of three phases: a pre-randomization screening phase (2 weeks); a double-blind treatment phase (18 weeks); and an open-label extension phase (52 weeks). The double-blind treatment phase was further divided into a titration period of 4 weeks, a maintenance period of 12 weeks, and a follow-up period of 2 weeks. Patients who met all inclusion criteria were randomized in a 1:1 ratio to receive either study medication or placebo during double-blind treatment (16 weeks). After randomization into the double-blind treatment phase, oxcarbazepine or placebo was initiated at 300 mg/day, and increased 3 days later to 300 mg twice a day (600 mg/day). After this, oxcarbazepine was titrated as tolerated up to a maximum target dose of 900 mg twice a day (1800 mg/day) in increments of 300 mg every 5 days over the 4-week titration period. During the remaining 12 weeks of the study (maintenance period), oxcarbazepine treatment remained at the dose reached by day 28. Patient response to the following three questions from the daily sleep questionnaire were used to assess disturbances of sleep over double-blind treatment: 1) Did your pain delay you in getting to sleep last night? 2) Did your pain awaken you from sleep more than one time last night? 3) Did you feel rested after sleep this morning?

The mean proportion of days that patients were awakened during the night due to pain was lower in oxcarbazepine-treated patients compared with the placebo group (31% vs 49% of study days; P=0.02) (Table 1). In addition, a lower proportion of oxcarbazepine-treated patients experienced delays in getting to sleep and a greater proportion of oxcarbazepine-treated patients felt rested after sleep in comparison with placebo-treated patients, but the differences did not achieve statistical significance.

TABLE 1
Therapeutic Effect and Sleep Questionnaire

| | ITT population | | |
|---|---|---|---|
| | | | P value (vs |
| | Oxcarbazepine | Placebo | placebo) |
| Sleep questionnaire | n = 68 | n = 76 | |
| % days during double-blind | | | |
| phase, mean (SD) | | | |
| Delayed getting to sleep | 28 (31) | 38 (38) | 0.09 |
| Awaken from sleep | 31 (29) | 49 (38) | 0.02 |
| Felt rested after sleep | 55 (34) | 46 (37) | 0.17 |

Abbreviations:
ITT, intent-to-treat;
SD, standard deviation

1. The use of oxcarbazepine for the manufacture of a pharmaceutical composition for the improvement of sleep in human patients suffering from chronic pain.

2. The use according to claim 1 wherein the pain is diabetic neuropathic pain.

3. The use according to claim 1 wherein oxcarbazepine is administered at a total dose between about 450 mg/day and about 2100 mg/day.

4. The use according to claim 1 wherein the sleep is improved by decreasing the frequency of being awakened from sleep due to pain.

5. The use according to claim 1 wherein the sleep is improved by decreasing the delay of getting to sleep due to pain.

6. The use according to any claim 1 wherein the sleep is improved by feeling rested after sleep.

7. A package comprising a pharmaceutical composition comprising as sole active ingredient oxcarbazepine together with instructions for improvement of sleep in human patients suffering from chronic pain.

8. A pharmaceutical composition comprising as sole active ingredient oxcarbazepine for the improvement of sleep in human patients suffering from chronic pain.

9. A method of improving sleep in human patients suffering from chronic pain, which comprises administering oxcarbazepine or a pharmaceutically acceptable salt thereof to said patient at a total dose in the range from about 1500 mg/day to about 2100 mg/day.

10. The method of claim 9 wherein a daily dose of from 1650 mg to 1950 mg is administered to the patient.

11. The method of claim 10 wherein the daily dose is about 1800 mg.

12. A method of improving sleep in human patients suffering from chronic pain, which comprises administering oxcarbazepine or a pharmaceutically acceptable salt thereof to said patient at a total dose in the range from about 900 mg/day to about 1500 mg/day.

13. The method of claim 12 wherein a daily dose of from 1050 mg to 1350 mg is administered to the patient.

14. The method of claim 13 wherein the daily dose is about 1200 mg.

15. A method of improving sleep in human patients suffering from chronic pain, which comprises administering oxcarbazepine or a pharmaceutically acceptable salt thereof to said patient at a total dose in the range from about 450 mg/day to about 900 mg/day.

16. The method of claim 15 wherein a daily dose of from 550 mg to 750 mg is administered to the patient.

17. The method of claim 16 wherein the daily dose is about 600 mg.

18. The method according to claim 9 wherein oxcarbazepine is administering on a twice daily schedule.

19. The method according to claim 1 wherein the patient is suffering from neuropathic pain.

20. The method according to claim 1 wherein the patient recruits from the Caucasian population.

21. The method of claim 19 wherein oxcarbazepine is administered in the form of a film-coated tablet.

22. A method of treating pain in a human patient, which comprises administering oxcarbazepine or a pharmaceutically acceptable salt thereof to said patient suffering from pain at a total dose in the range from about 1500 mg/day to about 2100 mg/day.

23. The method of claim 22 wherein the daily dose is about 1800 mg.

24. A method of treating pain in a human patient, which comprises administering oxcarbazepine or a pharmaceutically acceptable salt thereof to said patient suffering from pain at a total dose in the range from about 900 mg/day to about 1500 mg/day.

25. The method of claim 24 wherein the daily dose is about 1200 mg.

26. A method of treating pain in a human patient, which comprises administering oxcarbazepine or a pharmaceutically acceptable salt thereof to said patient suffering from pain at a total dose in the range from about 450 mg/day to about 900 mg/day.

27. The method of claim 26 wherein the daily dose is about 600 mg.

28. The method of claim 22 wherein the patient is suffering from diabetic neuropathic pain.

29. The method according to claim 22 wherein the patient recruits from the Caucasian population.

30. The use of oxcarbazepine for the manufacture of a pharmaceutical composition for the treatment of diabetic neuropathic pain.

31. A package comprising a pharmaceutical composition comprising as sole active ingredient oxcarbazepine together with instructions for the treatment of pain.
